(19) United States
(12) Patent Application Publication
(10) Pub. No.: US 2024/0124807 A1
(43) Pub. Date: Apr. 18, 2024

(54) METHODS FOR REDUCING WATER CONSUMPTION, CARBON EMISSIONS AND CHEMICAL USAGE

(71) Applicant: Wizso Limited, Belfast (GB)

(72) Inventor: Mark Fintan Gilligan, Belfast (GB)

(21) Appl. No.: 18/554,129

(22) PCT Filed: Apr. 7, 2022
(86) PCT No.: PCT/GB2022/050880

§ 371 Date: Oct. 5, 2023

(30) Foreign Application Priority Data
Apr. 8, 2021 (GB) 2105034.9

(51) Int. Cl. C11D 3/395; A61L 9/05; C11D 3/00; C11D 3/10; C11D 3/18; C11D 3/20; C11D 3/22; C11D 3/33; C11D 3/37; C11D 3/382; C11D 3/40; C11D 3/50; C11D 11/00; C11D 17/06; E03D 9/00

(52) U.S. Cl. CPC C11D 3/3953; A61L 9/05; C11D 3/0094; C11D 3/10; C11D 3/185; C11D 3/2079; C11D 3/2082; C11D 3/2086; C11D 3/225; C11D 3/33; C11D 3/3707; C11D 3/3776; C11D 3/382; C11D 3/40; C11D 3/507; C11D 11/0082; C11D 17/06; E03D 9/005

(57) ABSTRACT

A method for reducing water consumption in a toilet system comprises adding a water-soluble pellet to the toilet bowl prior to or following use of said toilet for urination, the pellet comprising a colourant and a fragrance for masking the colour and odour of urine residing in the toilet bowl, and preferably also disintegrant and/or an effervescent system that effervesces on contact with water, and refraining from flushing said toilet until after said toilet has been used at least one further time. The use of such water-soluble pellets serves to reduce the perceived need to flush the toilet after every use and drives positive change against wasteful water usage. The pellets are small, for example 8 mm or less in diameter and weigh 0.1-0.3 g, and can be stored in a re-sealable, refillable, reusable dispenser for personal or larger scale use. The method also serves to reduce carbon emissions and chemical usage arising from treatment of fresh and waste water.

### TECHNICAL FIELD

The present invention relates to a method of reducing water consumption in flushing toilet systems, as well as a method of reducing carbon emissions and chemical usage for water treatment, and to pellets or the like for use in said methods.

### BACKGROUND

Water is a precious resource; it is estimated that only about 0.3% of the Earth's water is fresh, drinkable water. Water wastage has been a worldwide issue for a long time. According to recent studies, flushing toilets represents the largest single use of indoor water consumption, amounting to roughly one third of the total household water consumed. A single toilet flush can use as much as 7.5 litres of water, an amount that many people in developing countries rely on for their entire daily needs.

Various means for reducing water consumption relating to toilet usage have been implemented. For example, water saving devices have been produced that work by reducing the volume of water normally retained in a toilet cistern, such as the so-called ‘Hippo’ bag which is placed in the cistern to reduce its capacity and hence reduce the volume of water that is stored and released into the toilet bowl on flushing. Dual-flush toilets that include a mechanism to flush different amounts of water, typically a smaller volume for flushing liquid waste and a larger volume for flushing solid waste, are in common usage.

The use of cleaning preparations to maintain hygiene within the toilet bowl is well known. For example, toilet blocks can be placed either in the cistern itself or in the path of the water flowing from the cistern for discharging cleaning compounds into the toilet bowl during flushing. Other toilet cleaning products, such as bleach preparations, gels or effervescent tablets, may be used periodically to clean the toilet bowl, often in conjunction with a toilet brush to ensure that the cleaning product is brought into contact with the entire bowl. Once such cleaning products have been introduced, and the toilet bowl brushed if desired, it is common to flush the toilet afterwards. Accordingly, while facilitating good hygiene, the use of such cleaning products does not contribute to any overall reduction in water consumption.

It is an object of the present invention to provide a method for reducing water consumption in a toilet system. It is a further object of the present invention to provide suitable products for use in such a method.

### SUMMARY OF THE INVENTION

From a first aspect, the present invention resides in a method for reducing water consumption in a toilet system, said method comprising adding a water-soluble pellet to the toilet bowl following use of said toilet for urination, said pellet comprising a colourant and a fragrance for masking the colour and odour of urine residing in the toilet bowl, and refraining from flushing said toilet until after said toilet has been used at least one further time.

As will be understood, by addition of the pellet to the toilet bowl after urination the present method effectively creates the impression of cleanliness and hence removes a perceived need to flush the toilet system after every use. The subsequent user of the toilet, whether the same or a different person to the previous user, will encounter a toilet that looks clean and smells fresh, but it has not been necessary to flush the toilet to achieve this condition. Thus, the method of the invention enables substantial savings in the amount of water used in the toilet system throughout the day by removing or at least substantially reducing the frequency of flushing. By reducing water consumption, the application of the invention therefore also serves to reduce chemical usage required to process fresh water and in the treatment of waste water, as well as reducing associated carbon emissions.

From another aspect, the invention also resides in a pellet for use in a method of reducing water consumption in a toilet system, said pellet being water-soluble and comprising a colourant and a fragrance for masking the colour and odour of urine when added to a toilet bowl.

Expressed in another way, the present invention comprises use of a water-soluble pellet comprising a colourant and a fragrance in a method for reducing water consumption in a toilet system by adding said pellet to the toilet bowl following urination instead of flushing said toilet.

The pellet according to the invention can be directly put into the toilet bowl just before or after the toilet has been used. The pellet will dissolve on contact with the urine-containing water in the toilet bowl, release a fragrance to cover the urine odour from the toilet, and turn the water into a colour that masks the colour of urine. As the pellet dissolves, it can be heard, smelt, and seen by the user so that he/she can be confident that the toilet is left recognisably clean for a subsequent user. Accordingly, many unnecessary flushes can be avoided, and substantial volumes of water may therefore be saved.

In this way, the present invention provides a solution for toilet systems that may be used to drive behavioural change regarding fresh water usage in the home, at work and in hotels and other hospitality venues. The invention therefore provides both environmental and economic benefits.

It will be understood that use of a second or further pellets is also contemplated after subsequent uses of the toilet, thereby enabling two or more flushing cycles to be avoided and further increasing the overall volume of water saved.

While the invention is primarily applicable for use with flushing toilet systems, and it is indeed the main aim of the invention to reduce the frequency of flushing thereby to reduce water consumption, it will be appreciated that the invention may be used in other types of toilet facility. For example, the invention may be used in relation to portable toilets or simple buckets, such as those would be used when camping.

The term “pellet” as used herein is intended to embrace products that may otherwise be described as tablets, pills, capsules, caplets, pilules and the like, and as such are capable of being dispensed individually, as a single dose, as required. While the term “pellet” is conventionally used to describe a small, rounded or tubular mass, its use herein is not limited to any particular shape. Use of a water-soluble pellet provides a convenient means of dispensing the required amount of colouring and fragrancing components into the toilet bowl, and they are easy to store and transport.

The pellet used in the invention is water-soluble and may comprise an effervescent system that effervesces on contact with water, preferably an effervescent system based upon an acid and a carbonate. As the pellet is added to water in the toilet bowl it begins to dissolve, and the acid and carbonate react to release carbon dioxide, generating bubbles within the water. Indeed, the bubbles formed may enhance the dissolution process, so that the rate of dissolution increases. The acid may be selected from one or more of the following: citric acid, oxalic acid, tartaric acid, malic acid, adipic acid and fumaric acid. The carbonate is preferably selected from one or more of the following: sodium carbonate, sodium bicarbonate, potassium carbonate and potassium bicarbonate.

While the use of an effervescent system may be advantageous, the pellets according to the invention are not limited thereto and they may instead incorporate alternative systems for achieving rapid or instant release of the colourant and fragrance on contact with water, for example for use instead of an effervescent system or in addition thereto. Such alternative systems may, for example, comprise disintegrants, including so-called “superdisintegrants”, typically swelling on contact with water thereby distorting the pellet structure and helping break up the pellet. Examples of disintegrants include, but are not limited to, cellulose and derivatives thereof, such as carboxymethylcellulose, sodium croscarmellose and hydroxypropyl methylcellulose, starch or modified starches, such as sodium starch glycolate, alginic acid, and swelling clays, such as bentonite.

In an embodiment, the pellet according to the invention comprises both an effervescent system and a disintegrant. Preferably any effervescent or disintegrant system, or combination thereof, results in substantially total dissolution of the pellet within 90 seconds of coming into contact with water in the toilet bowl.

The pellet may comprise a colourant to mask the colour of urine. Colourants may be selected from liquid dyes or powder dyes; the latter being more generally referred to as pigments. Advantageously, the colourant is a water-soluble dye. The water-soluble dye may be a natural dye or a synthetic dye, however synthetic dyes are currently preferred since they generally offer greater intensity of colour than natural dyes. The colourant should preferably provide an impressive visual impact with the colour being sustainable for several hours, so that a subsequent user sees an apparently clean and fresh toilet even after the effervescing has subsided and the fragrance dissipated. One such colourant is a blue-coloured dye, such as “Brilliant Blue FCF” (typically denoted by E number E133) which is a triarylmethane dye consisting of disodium; 2-[[4-[ethyl-[(3-sulfonatophenyl) methyl] amino] phenyl]-[4-[ethyl-[(3-sulfonatophenyl) methyl] azaniumylidene]cyclohexa-2,5-dien-1-ylidene]methyl]benzenesulfonate. Other colourants that may be used include, but are not limited to, green-coloured dyes, such as sodium copper chlorophyllin, and purple-coloured dyes, such as mauveine.

The pellet may also comprise a fragrance to mask the odour of urine. The fragrance may be a natural or a synthetic fragrance and may, for example, be in the form of a liquid or powder. Natural fragrances, for example, may be based upon one or more essential oils, such as lemon, pine, lavender and rose essential oils.

Other functional ingredients may optionally be included in the pellet according to the invention. For example, the pellets may further comprise one or more components selected from, but not limited to foaming agents, bleaching agents, binders, fillers, water softeners and lubricants. Addition of an enzyme and/or surfactants to the pellet to enhance its cleaning ability is also contemplated within the scope of the invention. Inclusion of one or more biological agents, such as free and/or bacterial enzymes, may assist with biological cleaning and odour removal. For example, addition of free enzymes may provide almost instantaneous breakdown of odour-producing organic matter. Addition of protease, causing breakdown of proteinaceous material in urine, and breakdown of uric acid which may cause scaling around the toilet bowl, may be advantageous.

The use of a foaming agent as a component to enhance the effervescent system is advantageous, promoting the generation of a foam when the carbon dioxide is released into the water through reaction of the acid and carbonate. The foaming agent may comprise a surfactant, preferably selected from non-ionic and anionic surfactants, such as alkyl sulphates, alkyl ether sulphates, alkyl sulphonates. Examples of preferred surfactants include sodium laureth sulphate, sodium lauryl sulphate, and coco-glucoside, with sodium lauryl sulphate being especially preferred.

The pellet of the invention may include a bleaching agent, such as a solid bleaching agent, for example a persulphate, perborate or percarbonate, or a hypochlorite or peracid generator. Preferably the bleaching agent is sodium percarbonate, which hydrolyses on contact with water to produce hydrogen peroxide, and it has a good shelf-life as well as low toxicity. Milder, naturally occurring bleaching agents, such as citric acid, may in any event be included as a component of the effervescent system. The incorporation of a bleaching agent into the pellet not only aids decolorization of urine, but also helps sanitise and/or disinfect the toilet bowl. Accordingly, such pellets offer benefits not just in terms of masking the colour and odour of urine but may positively contribute to the hygiene of the toilet bowl.

The pellet of the invention may further include one or more binders, for example in the form of sugars, natural binders and/or synthetic/semisynthetic binders, all of which are commonly referred to as “tablet binders”. Isomalt (a combination of glucose and mannitol), D-sorbitol and polyvinyl pyrrolidone, are examples of preferred binders, especially for dry granulation where the pellet composition is in a directly compressible form. Polyvinyl pyrrolidone in ethanol (10% PVP) may also be used as a binder for wet granulation of the pellet composition, as may polyethylene glycol, such as PEG 400, which is liquid under ambient conditions may similarly be used as a binder for wet granulation.

Alternatively, ethanol may be used to form the pellet by wet granulation, serving to wet the powder and help “bind” the components without the need for a conventional binder, and use of ethanol has been found to result in faster dissolving pellets than those containing sorbitol or PVP in ethanol.

Use of lubricants in the pellets of the invention is also beneficial for providing improved powder processing properties of the pellet formulation, especially to improve flowability into the pellet press as well as easing ejection of the pellet after pressing. Sodium benzoate and magnesium stearate are examples of lubricants for use in the pellets of the invention.

The pellet of the invention may also incorporate a water softener. Water softeners are useful to reduce water hardness and hence discourage build-up of limescale in the toilet bowl. In an embodiment, the pellet of the invention may comprise a chelating agent as a water softener, acting by removing minerals such as calcium and magnesium. As an example, aspartic acid and/or polyaspartic acid (PASA) may be used as a chelating water softener in the pellets of the invention. PASA is biodegradable.

The pellet of the invention may optionally include one or more fillers. Any such filler should preferably be water-soluble. Sodium chloride is an example of a filler that may be incorporated into the pellet composition. Alternatively, the aforementioned binders may also act as fillers/binders for the pellet compositions.

Typically, the effervescent system comprises from about 65 to about 95 wt % based on the weight of the pellet, preferably from about 70 to 90 wt %. For example, the effervescent system may comprise from 15 to 50 wt % citric, adipic and/or fumaric acid, and from 35 to 60 wt % sodium bicarbonate. Within the effervescent system, it is preferred that acid is present at least in a stoichiometric equivalent to (bi)carbonate, but more preferably in stoichiometric excess with respect to the (bi)carbonate. In this way, excess acid may serve to deter deposition of limescale within the toilet bowl.

The pellet may optionally comprise a disintegrant in an amount up to about 10 wt %. Preferably the disintegrant is included in an amount of from about 4 to 8 wt %, most preferably alongside an effervescent system, for achieving rapid disintegration of the pellet on contact with water.

The pellet further comprises a fragrance, which may be a blend, in an amount of from about 0.5 to about 6 wt %, preferably from about 1 to 5 wt %, and typically about 4 wt %. Similarly, the pellet further comprises a colourant, which may be a colourant blend, in an amount of from about 0.5 to about 5 wt %, preferably from about 1 to 4 wt %, and typically about 3 wt %.

A surfactant, acting as a foaming agent in conjunction with the effervescent system and/or disintegrant, may optionally be present in the pellet in an amount of from about 0.1 to about 5 wt %, preferably from about 0.25 to about 1 wt %, more preferably about 0.5 wt %.

A binder may optionally be present in an amount up to about 10 wt %, such as from about 1 to about 8 wt %, preferably from about 2 to 6 wt %, and more preferably about 5 wt %, based on the weight of the pellet.

A bleaching agent may optionally be present in an amount up to about 15 wt %, such as from about 0.5 to about 10 wt %, typically from about 4 to about 8 wt %, based on the weight of the pellet.

A lubricant may optionally be present in an amount up to about 8 wt %, such as from about 0.5 to about 8 wt %, typically about 5 wt %, based on the weight of the pellet.

A water softener, such as a chelating agent, may optionally be present in an amount up to about 2 wt %, such as from about 0.1 to 2 wt %, preferably from about 0.5 to 1 wt %.

The pellet for use in the present invention is preferably formed in a compact size so that a supply of such pellets can be readily stored and/or transported, whether on an industrial scale for commercial use, or at an individual consumer level when intended for personal use. It is proposed for example, that dispensers containing a bulk supply of pellets may be installed in public toilets, such as in schools, hotels and at work, and smaller dispensers are made available for personal use, for example allowing an individual to carry a supply in their pocket or handbag, or to have available in the bathroom at home.

Of course, it is also necessary for any pellet to have a volume sufficient to deliver the desired effects, namely to release a depth of colour and fragrance to mask the colour and odour of urine, when dispensed into the toilet bowl, and preferably also be seen to effervesce. On the other hand, it is preferable to limit the volume of the pellet to that which is required to achieve the desired effects without unnecessary excess. It has been found that a pellet weighing from about 0.1 to about 2 g, preferably from about 0.1 to about 1 g, and most preferably from 0.1 to 0.5 g, such as about 0.25 g, is particularly suitable to deliver the requisite properties for the purpose of the present invention. Such a pellet may be formed with a diameter in the region of from about 2 mm to 10 mm, with a diameter of from about 6 mm to 9 mm being preferred. In an embodiment, the pellet for use in the method(s) of the invention comprises an effervescent system, a disintegrant, and a bleaching agent, in addition to a fragrance and a colourant, with an excipient comprising one or more of a binder, foaming agent, water softener and lubricant. The relative proportions of each of the components may be within the ranges hereinbefore described. The pellet may have a weight of 2 g or less, preferably from 0.1 to 0.5 g.

The pellet may be formed in a conventional shape or profile, such as round, oval, lozenge or capsule shaped, or in a more complex shape, such as diamond or heart shaped. The shape and profile may be selected to enhance rate of dissolution when placed in water, or to provide a distinctive image to enhance brand awareness. In an embodiment, the dimensions and weight of the pellet may be selected so that when dropped into the toilet bowl, the buoyancy of the pellet causes it to rise to, the water's surface with the result that the colourant disperses across the surface, accelerated by the action of the effervescent system and/or disintegrant. Thus, the colouration of the water is observed more swiftly than if the pellet were to sink and remain on the base of the toilet bowl while it effervesces or otherwise disintegrates. In this regard, the pellet may be formed with substantially flat, or slightly convex, upper and lower surfaces and of a diameter that exceeds its thickness, such as disc-shaped or similar. Forming the pellet with slightly convex upper and lower surfaces may be beneficial in releasing the pellet during moulding thereof.

Since the pellet of the invention is water-soluble, exposure to moisture in the air may result in degradation and/or weight loss over a period. In this regard it has been found that weight loss is less in pellets of greater diameter. However, suitable storage conditions can inhibit possible degradation from moisture, and therefore it is desirable that supplies of the pellet are stored in a sealed or re-sealable container to reduce or avoid inadvertent exposure of the pellet to moisture. Thus, after production, the pellets may be transferred into a sealed compartment, for example into a sealed pouch or loose in a re-sealable dispenser. When stored loose, use of a desiccant, such as a silica gel sachet, within the container may be beneficial.

Pellets according to the invention may be prepared by any suitable method. It is preferable that the components of the pellet are combined in an anhydrous environment to avoid premature degradation and/or reaction between the components prior to their intended use.

One method of formulating the pellets involves combining the solid components, especially in powder form, to form a mixture, and grinding the mixture to provide a powder-mix. For example, components of the effervescent system and/or disintegrant if used, such as acid, carbonate, percarbonate and surfactant, together with a colourant may be supplied as solid powders and ground together to form a powder-mix. Subsequently, binder and fragrance may be added to the powder-mix and blended to form a pre-mix. The pre-mix may optionally be further blended with a lubricant. After all the desired components have been combined to form a substantially homogenous blend, the blend may then be vacuum-dried and sieved, prior to press-moulding to form the pellets.

Vacuum drying may suitably be conducted at a temperature in the range of from 50 to 80° C., preferably at about 60° C. The dried formulation may be passed through any suitable sieve to obtain fine particles, for example, through a 100-mesh sieve.

The press-moulding, for example using a conventional tablet press, may be performed at a predetermined compression for achieving the desired density in the formed pellet. As hereinbefore mentioned, the density of the pellet (based upon weight, dimensions and degree of compression) is preferably selected so that the pellet floats or rises to the surface when dropped in water.

### BRIEF DESCRIPTION OF THE DRAWINGS

One or more embodiments of the invention will now be described with reference to the accompanying drawings, in which;

FIG. 1 shows a pellet dispenser for storing and dispensing loose pellets for use in the methods of the present invention; and

FIG. 2 shows a personal pellet dispenser for storing and dispensing loose pellets for use in the methods of the present invention.

### DETAILED DESCRIPTION OF THE INVENTION

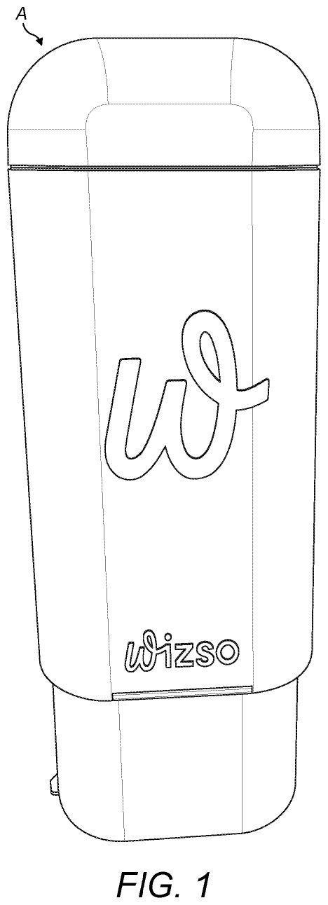

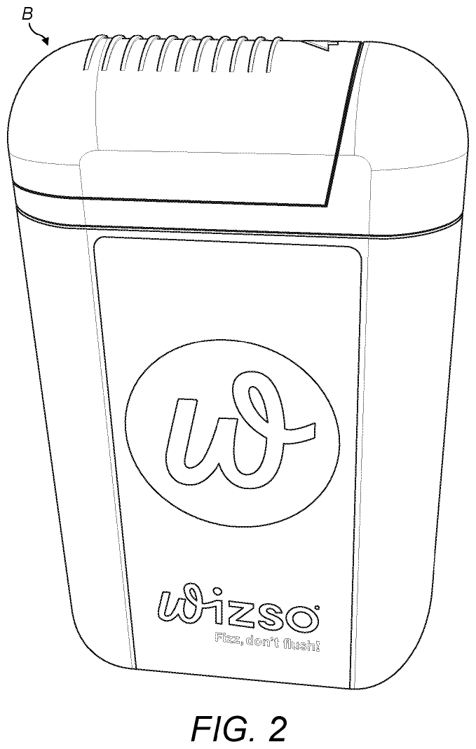

FIGS. 1 and 2 of the drawings illustrate examples of pellet dispensers that are useful for storing and dispensing pellets according to the present invention. The pellet dispensers shown are refillable and reusable, as well as being re-sealable to prevent undue exposure to moisture in the atmosphere.

The pellet dispenser A of FIG. 1 is of a size to accommodate bulk supplies of pellets which can be dispensed singly therefrom. The dispenser A is suitable for example to be kept at home in the bathroom, or installed for use in a public space, such as in public conveniences, in offices, hotels or schools and the like. The dispenser A can be free standing, wall mounted or fixed to the toilet. Optionally, the dispenser A may further accommodate a desiccant, such as a silica gel, which may be periodically replaced, for example when restocking with pellets.

The pellet dispenser B shown in FIG. 2 is suitable for personal use and is of a size that can be comfortably carried in a user's pocket or handbag. Such a dispenser may be similar to those already known for dispensing mints or artificial sweeteners.

The invention will now be further illustrated by way of example, with reference to the following non-limiting Examples.

### Example 1

For comparison, Pellets A, B and C were prepared from the following compositions based upon different “binders” for wet or dry granulation, each being a 6 mm diameter pellet weighing 0.15 g:

### Pellet A:

49 wt % citric acid, 42 wt % sodium bicarbonate, 5 wt % ethanol, 2 wt % rose oil and 2 wt % Brilliant blue dye.

### Pellet B:

49 wt % citric acid, 42 wt % sodium bicarbonate, 5 wt % of 10% polyvinyl pyrrolidone in ethanol, 2 wt % rose oil and 2 wt % Brilliant blue dye.

### Pellet C:

49 wt % citric acid, 42 wt % sodium bicarbonate, 5 wt % D-sorbitol, 2 wt % rose oil and 2 wt % Brilliant blue dye.

Each of Pellets A, B and C were used in accordance with the method of the invention, namely by adding to the toilet bowl following urination therein. Pellet A, based on ethanol “binder”, displayed the best performance, having good rigidity and superior dissolving ability as compared to Pellets B and C. Pellet B, based upon PVP binder in ethanol, dissolved more slowly and Pellet C, based upon D-sorbitol binder, had inferior colouration ability when dissolved as compared to Pellets A and B.

### Example 2

For comparison, Pellets D, E and F were prepared with the following compositions based upon different effervescent systems, specifically using different acids:

### Pellet D:

49 wt % citric acid, 42 wt % sodium bicarbonate, 5 wt % ethanol, 2 wt % rose oil and 2 wt % Brilliant blue dye.

### Pellet E:

41 wt % oxalic acid, 50 wt % sodium bicarbonate, 5 wt % ethanol, 2 wt % rose oil and 2 wt % Brilliant blue dye.

### Pellet F:

25 wt % citric acid, 20 wt % oxalic acid, 46 wt % sodium bicarbonate, 5 wt % ethanol, 2 wt % rose oil and 2 wt % Brilliant blue dye.

Each of Pellets D, E and F were used in accordance with the method of the invention. Pellet D based upon citric acid with sodium bicarbonate as effervescing agents displayed a superior “fizzing” effect as compared to Pellets E and F.

### Example 3

Pellets having the following composition were prepared according to the steps described:

| | Ingredient | wt % |
|---|---|---|
| Acid | Citric Acid | 20 |
| Carbonate | Sodium Bicarbonate | 55 |
| Binder | PEG-400 | 5 |
| Bleaching Agent | Sodium Perborate | 8 |
| Lubricant | Sodium Benzoate | 5 |
| Fragrances | Lavender Essential Oil | 2 |
| | Limonene | 2 |
| Colorant | Blue E133 | 3 |
| Total | | 100 |

1. Sodium benzoate, supplied in crystalline form, is ground or milled into a powder.

2. Solid ingredients in powder form including sodium bicarbonate, sodium perborate, sodium benzoate and the colorant were combined to obtain a powder-mix.

3. Liquid ingredients including PEG-400 (a low molecular weight grade of polyethylene glycol), essential oil and limonene were then added to the powder-mix and blended to obtain a uniformly coloured pre-mix.

4. Citric acid in powder form was added to the pre-mix and then blended to obtain a uniform pellet mix.

5. The pellet mix is fed to a tablet press and metered into the mould cavities of the press and compressed to form the pellets. For example, the mould cavities may conveniently be formed by two punches and a die, with the punches pressed together with force to fuse the pellet mix and form the pellets.

6. The formed pellets are ejected from the mould and packaged in bulk in a sealable container.

In the above method, the formed pellets have a weight of 0.25 g and a diameter of 6 mm. The sealable container is provided with a silica gel sachet to absorb any moisture from the air and prevent premature degradation of the pellets.

### Example 4

Pellets having the following composition were formed by press moulding:

| | Ingredient | wt % |
|---|---|---|
| Acid | Fumaric Acid | 32 |
| Carbonate | Sodium Bicarbonate | 46 |
| Disintegrant | Carboxy methylcellulose | 6 |
| Binder/Lubricant | PVP, Isomalt, Magnesium | 4 |
| | Stearate | |
| Bleaching Agent | Sodium Perborate | 7.5 |
| Water Softener | Aspartic Acid | 0.5 |
| Fragrance | Fragranced Powder | 1.2 |
| Colorant | Blue E133 | 2.8 |
| Total | | 100 |

The present invention as described herein provides a solution to unnecessary and/or excessive use of water in toilet systems and drives positive behavioural change by increasing awareness of the environmental and cost benefits in using the pellets to reduce water consumption and the significant carbon emissions associated with fresh water treatment and waste water treatment. In addition, there is also a reduction in the use of water treatment chemicals involved in the treatment of both fresh water prior to use and waste water after use. By consciously taking action to reduce water consumption in the context of flushing of toilets, this helps increase overall awareness of water being a precious resource and therefore may indirectly influence behaviour in other aspects of life.

Although particular embodiments of the invention have been described herein in detail, it should be understood that this has been done by way of example and for the purpose of illustration only. The aforementioned embodiments are not intended to be limiting with respect to the scope of the appended claims, which follow. It is contemplated by the inventors that various substitutions, alterations and modifications may be made to the invention without departing from the scope of the invention as defined by the claims.

1. A method for reducing water consumption in a toilet system, said method comprising adding a water-soluble pellet to the toilet bowl prior to or following use of said toilet for urination, said pellet comprising a colourant and a fragrance for masking the colour and odour of urine residing in the toilet bowl, and refraining from flushing said toilet until after said toilet has been used at least one further time.

2. A pellet for use in a method of reducing water consumption in a toilet system, said pellet being water-soluble and comprising a colourant and a fragrance for masking the colour and odour of urine when added to a toilet bowl.

3. Use of a water-soluble pellet comprising a colourant, and a fragrance for reducing water consumption in a toilet system by adding said pellet to the toilet bowl prior to or following urination instead of flushing said toilet.

4. A method for reducing carbon emissions and/or reducing chemical usage in water treatment, said method comprising reducing water consumption in a toilet system by adding a water-soluble pellet to the toilet bowl prior to or following use of said toilet for urination, said pellet comprising a colourant and a fragrance for masking the colour and odour of urine residing in the toilet bowl, and refraining from flushing said toilet until after said toilet has been used at least one further time.

5. The methods, pellet or use according to any one of claims 1 to 4, wherein the toilet system is a flushing toilet system.

6. The methods, pellet or use according to any one of claims 1 to 5, wherein the pellet further comprises an effervescent system that effervesces on contact with water.

7. The methods, pellet or use according claim 6, wherein the effervescent system comprises an acid component and a carbonate component for releasing carbon dioxide when the acid and carbonate together come into contact with water.

8. The methods, pellet or use according to claim 7, wherein the acid is selected from citric acid, oxalic acid, tartaric acid, adipic acid, malic acid and fumaric acid, or any combination thereof.

9. The methods, pellet or use according to claim 7 or 8, wherein the carbonate is selected from sodium carbonate, sodium bicarbonate, potassium carbonate and potassium bicarbonate, or any combination thereof.

10. The methods, pellet or use according to any one of the preceding claims, wherein the pellet further comprises a disintegrant.

11. The methods, pellet or use according to claim 10, wherein the disintegrant is selected from cellulose, a cellulose derivative, sodium croscarmellose, hydroxypropyl methylcellulose, carboxy methylcellulose, starch, modified starch, alginic acid and a swelling clay.

12. The methods, pellet or use according to any one of the preceding claims, wherein the pellet comprises an effervescent system and a disintegrant.

13. The methods, pellet or use according to any one of the preceding claims, wherein the pellet further comprises a bleaching agent, preferably the bleaching agent is selected from a persulphate or percarbonate.

14. The methods, pellet or use according to any one of the preceding claims further comprising one or more of a binder, lubricant, enzyme, foaming agent and water softener.

15. The methods, pellet or use according to claim 14, wherein the binder is selected from ethanol, polyvinylpyrrolidone, isomalt and D-sorbitol, or any combination thereof.

16. The methods, pellet or use according to any one of the preceding claims, wherein the colourant comprises a liquid or powder dye, preferably a water-soluble dye, such as Brilliant Blue FCF or other possible colourants.

17. The methods, pellet or use according to any one of the preceding claims, wherein the fragrance comprises an essential oil, preferably a lemon oil, pine oil, lavender oil or rose oil.

18. The methods, pellet or use according to any one of the preceding claims, wherein the pellet comprises about 65 to about 95 wt % of an effervescent system, about 0.5 to about 6 wt % of fragrance, about 0.5 to about 4 wt % of colourant, and optionally further comprising up to about 10 wt % binder, up to about 15 wt % bleaching agent, up to about 10 wt % disintegrant, about 0.1 to about 5 wt % surfactant and about 0.5 to about 2 wt % lubricant, all wt % based on the total weight of the pellet.

19. The methods, pellet or use according to any one of the preceding claims, wherein the pellet has a density less than the density of water such that it floats, or rises to the surface, when initially added to the toilet bowl.

20. The methods, pellet or use according to any one of the preceding claims, wherein the pellet has a weight in the range of from 0.02 to about 2 g, preferably from about 0.05 to about 1 g, and most preferably from 0.1 to 0.5 g.

21. The methods, pellet or use according to any one of the preceding claims, wherein the pellet is formed with a diameter in the region of from about 6 mm to 10 mm, preferably from about 6 mm to 9 mm.

22. The methods, pellet or use according to any one of the preceding claims, wherein a plurality of said pellets are stored in bulk a re-sealable container adapted to dispense individual pellets when required for use or in a blister pack.

23. A method of manufacturing a water-soluble, effervescent pellet for use in a toilet system to reduce the frequency of flushing after toilet use, said method comprising combining an acid and a carbonate, a colourant and optionally a surfactant and/or bleaching agent and/or disintegrant, each in solid form and grinding to form a powder-mix, adding a binder and fragrance and further blending to form a substantially homogeneous pre-mix, optionally adding a lubricant to said pre-mix, and press-moulding up to about 2 g, and preferably from about 0.05 to 1 g, of said homogeneous pre-mix in a mould having an internal diameter of about 10 mm or less, and preferably from about 3 to 6 mm, to form said pellet.

24. The method of claim 23, wherein the homogeneous pre-mix is vacuum dried and passed through a sieve, such as a 100-mesh sieve, to form fine particles prior to press moulding.

25. The method of claim 23 or claim 24, wherein said method is carried out under anhydrous conditions.
